SEQUENCE LISTING (4 sequences)

SEQ ID NO: 1 - protein, 157 aa, Homo sapiens
DLGKKLLEAARAGQDDEVRILMANGADVNANDRNGVTPLHLAADKGHLEIVEVLLKTGADVNAIDIMGATPLHLAAAHGHLEIVEVLLKAGADVNAMDHKGFTPLHLAAWRGHLEIVEVLLKHGADVNAQDKFGKTPFDLAIDNGNEDIAEVLQKAA

SEQ ID NO: 2 - DNA, 471 bp, Homo sapiens
gacctgggtaagaaactgctggaagctgctcgtgctggtcaggacgacgaagttcgtatcctgatggcaaacggtgctgacgttaacgctaacgaccgtaacggtgttactccgctgcacctggctgctgacaaaggtcacctggaaatcgttgaagttctgctgaaaaccggtgctgacgttaacgctatcgacatcatgggtgctactccgctgcacctggctgctgctcatggtcacctggaaatcgttgaagttctgctgaaggccggcgctgacgttaacgctatggaccataaaggtttcactccgctgcacctggctgcttggcgtggtcacctggaaatcgttgaagttctgctgaagcacggcgccgacgttaacgctcaggacaaattcggtaagactccgttcgacctggctatcgacaacggtaacgaggacatcgctgaagttctgcagaaagctgct

SEQ ID NO: 3 - protein, 182 aa, Homo sapiens
MRGSHHHHHHHHGSDLGKKLLEAARAGQDDEVRILMANGADVNANDRNGVTPLHLAADKGHLEIVEVLLKTGADVNAIDIMGATPLHLAAAHGHLEIVEVLLKAGADVNAMDHKGFTPLHLAAWRGHLEIVEVLLKHGADVNAQDKFGKTPFDLAIDNGNEDIAEVLQKAAKLNDYKDDDDK

SEQ ID NO: 4 - DNA, 546 bp, Homo sapiens
atgagaggatcgcatcaccatcaccatcaccatcacggatccgacctgggtaagaaactgctggaagctgctcgtgctggtcaggacgacgaagttcgtatcctgatggcaaacggtgctgacgttaacgctaacgaccgtaacggtgttactccgctgcacctggctgctgacaaaggtcacctggaaatcgttgaagttctgctgaaaaccggtgctgacgttaacgctatcgacatcatgggtgctactccgctgcacctggctgctgctcatggtcacctggaaatcgttgaagttctgctgaaggccggcgctgacgttaacgctatggaccataaaggtttcactccgctgcacctggctgcttggcgtggtcacctggaaatcgttgaagttctgctgaagcacggcgccgacgttaacgctcaggacaaattcggtaagactccgttcgacctggctatcgacaacggtaacgaggacatcgctgaagttctgcagaaagctgctaagcttaatgactacaaggatgacgacgacaag

(19) United States
(12) Patent Application Publication
(10) Pub. No.: US 2024/0018265 A1
(43) Pub. Date: Jan. 18, 2024

(54) HDAC6 BINDING PROTEINS AND THEIR ANTI-VIRAL USE

(71) Applicants: FRIEDRICH MIESCHER INSTITUTE FOR BIOMEDICAL RESEARCH, Basel (CH); UNIVERSITY OF ZURICH, Zurich (CH)

(72) Inventors: Patrick MATTHIAS, Basel (CH); Longlong WANG, Basel (CH); Andreas PLÜCKTHUN, Zurich (CH)

(21) Appl. No.: 18/256,817

(22) PCT Filed: Dec. 8, 2021
(86) PCT No.: PCT/IB2021/061460

§ 371 Date: Jun. 9, 2023

(30) Foreign Application Priority Data
Dec. 11, 2020 (EP) 20213494.6

(51) Int. Cl. C07K 16/40; C12N 9/80

(52) U.S. Cl. CPC C07K 16/40; C12N 9/80; C12Y 305/01098; C07K 2317/76; C07K 2317/569

(57) ABSTRACT

The present application provides a recombinant binding protein that specifically binds to HDAC6 and blocks the ubiquitin-engaging zinc finger domain of HDAC6.

### FIELD OF THE INVENTION

The present invention provides a new reagent and method for the treatment of virus infections.

### BACKGROUND OF THE INVENTION

The coronavirus disease 2019 (COVID-19) pandemic caused by the severe acute respiratory syndrome coronavirus 2 (SARS-CoV-2) which has taken the world by surprise exemplifies how viruses represent a continuous—but often underestimated—threat to human health. Beyond the new SARS-CoV-2 virus, RNA viruses such as influenza A virus (IAV) and coronaviruses come seasonally and affect every year millions of people worldwide. Viruses have evolved a multitude of highly specific and unique mechanisms that intersect with cellular pathways, often to favor the infection. One such pathway is ubiquitination, the process by which the small 76 amino acid cellular protein ubiquitin (Ub) is used to generate a variety of polymeric chains that can be post-translationally conjugated to proteins. Depending on which lysine residues (K48, K63 . . . ) are used for branching, the Ub polymers are structurally different and have distinct functions. By modulating protein function (e.g. localization, trafficking . . . ) or fate (e.g. degradation), ubiquitination impinges on most aspects of cellular metabolism. Proteins ubiquitinated by K48-branched chains are targeted for degradation by the ubiquitin proteasome system (UPS) (reviewed in (Komander and Rape, 2012)). Viruses often depend on the UPS (Isaacson and Ploegh, 2009): proteasome inhibitors block productive IAV entry and impact the replication of various virus classes (reviewed in (Rudnicka and Yamauchi, 2016)). For both IAV entry and cellular granules formation the Ub-interacting deacetylase HDAC6 is important. HDAC6 is a mostly cytoplasmic lysine deacetylase with unique properties: it has tandem catalytic domains (CD) which are organized in a pseudo-two-fold symmetric structure (Miyake et al., 2016) and also a conserved zinc finger domain (ZnF—UBP, hereafter ZnF) with homology to the Ub binding domain of deubiquitinases (Hook et al., 2002; Seigneurin-Berny et al., 2001). The main substrates of HDAC6 are tubulin (Hubbert et al., 2002; Zhang et al., 2003), and also the chaperone HSP90 (Kovacs et al., 2005), cortactin (Zhang et al., 2007) or the RNA helicase DDX3X (Saito et al., 2019). By regulating the level of tubulin and cortactin acetylation HDAC6 influences microtubule dynamics, cytoskeletal trafficking and cellular motility (Boyault et al., 2007). A variety of HDAC6-specific small molecule inhibitors have been developed which target its deacetylase activity and have shown efficacy in some cancer models (Brindisi et al., 2019; Cosenza and Pozzi, 2018; Mishima et al., 2015). In many cases the biological functions of HDAC6 depend, beyond the catalytic activity, on the ZnF domain which binds with high affinity to unanchored Ub chains via their C-terminal diglycine-GG motif (Boyault et al., 2006; Ouyang et al., 2012): formation of SGs and aggresomes requires an intact HDAC6 ZnF domain (Kawaguchi et al., 2003; Kwon et al., 2007). Likewise, infection by IAV only proceeds normally when the HDAC6 ZnF domain is functional; mutation of the HDAC6 ZnF domain so as to impair Ub chains recruitment strongly reduces the capacity of IAV to uncoat its capsid and release its nucleic acids (Banerjee et al., 2014). Many viruses carry and need Ub for infectivity (Gustin et al., 2011); the IAV viral capsid recapitulates key aspects of the aggresome pathway by bringing along unanchored Ub chains (Artcibasova et al., 2020, submitted). Moreover, this pathway and its core components (HDAC6 and Ub) have been very recently shown to be an essential part of the inflammasome activation (Magupalli et al., 2020). Thus, infection by IAV (and possibly other viruses) as well as cellular processes that are part of the stress or inflammatory response need the interaction between unanchored Ub chains and HDAC6; targeting this interaction may therefore be of great therapeutic interest.

### SUMMARY OF THE INVENTION

Previously, some of the present inventors found that a therapeutically effective amount of a modulator of the ubiquitin-binding property of HDAC6 could be used to treat virus infections. While trying to generate designed ankyrin repeat proteins (DARPins) and nanobodies inhibiting the ubiquitin-binding property of HDAC6, the inventors realised that the search for such agents is not straightforward. The inventors used purified human HDAC6 ZnF domain to identify DARPINS and nanobodies binding specifically to this domain. Among the numerous binding molecules identified as binding specifically to human HDAC6 ZnF domain, only one single DARPin, called F10 and having the amino acid sequence of SEQ ID NO:1, was identified as having the desired biological property of blocking the ubiquitin-engaging zinc finger domain of HDAC6.

The present invention hence provides recombinant binding protein comprising at least 90 consecutive amino acids of SEQ ID NO:1, wherein said recombinant binding protein specifically binds to HDAC6 and blocks the ubiquitin-engaging zinc finger domain of HDAC6. In one embodiment, the recombinant binding protein of the invention comprises an ankyrin repeat domain corresponding to amino acids 25 to 129 of SEQ ID NO:1. In another embodiment, the recombinant binding protein of the invention comprises an ankyrin repeat domain corresponding to amino acids 31 to 123 of SEQ ID NO:1. The recombinant binding protein of the invention can comprises 90, 95, 100, 105, 110, 115, 120, 125, 130,135, 140, 145, 150, 155, or 157 consecutive amino acids of SEQ ID NO:1, In one embodiment, the recombinant binding protein of the invention comprises at least 120 consecutive amino acids of SEQ ID NO:1. In one embodiment, the recombinant binding protein of the invention comprises an ankyrin repeat domain corresponding to amino acids 1 to 129 of SEQ ID NO:1. In another embodiment, the recombinant binding protein of the invention comprises an ankyrin repeat domain corresponding to amino acids 1 to 123 of SEQ ID NO:1. In one embodiment, the recombinant binding protein of the invention comprises an ankyrin repeat domain corresponding to amino acids 25 to 157 of SEQ ID NO:1. In another embodiment, the recombinant binding protein of the invention comprises an ankyrin repeat domain corresponding to amino acids 31 to 157 of SEQ ID NO:1. In one embodiment, the recombinant binding protein of the invention comprises SEQ ID NO:1. The present invention also provides a recombinant binding protein that competes for binding to HDAC6 with the recombinant binding protein described in this paragraph and blocks the ubiquitin-engaging zinc finger domain of HDAC6. The term “compete for binding” means the inability of two different recombinant binding proteins to bind simultaneously to the same target, while both are able to bind to this target individually, and can be routinely assessed by the practitioner in the art.

In some embodiments, the recombinant binding protein of the invention binds to HDAC6 with a K_{D} below 4 μM. As well known to the skilled person, the equilibrium dissociation constant (K_{D}) is the basic parameter to evaluate the binding property of the drug-receptor and can be determined by, a variety of analytical methods, including radioligand binding assay, surface plasmon resonance method, fluorescence energy resonance transfer method, affinity chromatography, and isothermal titration calorimetry.

The present invention also provides an isolated nucleic acid encoding a recombinant binding protein according to any of the preceding claims. In one embodiment, this isolated nucleic acid of the invention comprises the nucleic acid sequence of SEQ ID NO:2. For the example, the isolated nucleic acid molecule of the invention can have the nucleic acid sequence of SEQ ID NO:4, and code for the recombinant binding protein having the amino acid sequence of SEQ ID NO:3 (F10 with histidine and detection tags attached to it by BamH1 and HindIII sites, respectively).

The isolated nucleic acid of the invention can be integrated within an expression cassette and operatively linked to genetic elements, e.g. promoter, operator, regulator, or transient repressor, allowing and/or controlling its expression in target cells. Genetic elements allowing and/or controlling expression in target cells are well known to the skilled person. In some embodiments, the elements controlling the expression of the isolated nucleic acid allow for conditional expression. In addition, elements that will lead to the fusion of a cell-penetrating peptide, such as the HIV TAT protein, to the recombinant binding protein of the invention can also be used. The cell-permeable peptide can be fused to the DARPin at its C-terminus or at its N-terminus.

The present invention further comprises vectors comprising the expression cassette and isolated nucleic acids of the invention. In some embodiments, the vector can be a mRNA molecule. In some embodiments, this vector can be a viral vector, for instance the vector can be an AAV, a PRV or a lentivirus. In some embodiments, it is an AAV.

The recombinant binding proteins, isolated nucleic acids, expression cassettes or vectors of the invention can be used as medicament, for instance to treat viral infections. The present invention also provides pharmaceutical compositions comprising them, and optionally a pharmaceutical acceptable carrier and/or diluent.

The present invention provides methods for treating a viral infection in a subject characterised in that a therapeutically effective amount of recombinant binding proteins, isolated nucleic acids, expression cassettes, vectors or pharmaceutical compositions of the invention is administered to said subject. In some embodiments, the virus is an enveloped virus, for instance an influenza virus, a zika virus or an ebola virus.

The sequences of the invention are:

SEQ ID NO: 1
DLGKKLLEAARAGQDDEVRILMANGADVNANDRNGVTPLHLAADKGHLE
IVEVLLKTGADVNAIDIMGATPLHLAAAHGHLEIVEVLLKAGADVNAMD
HKGFTPLHLAAWRGHLEIVEVLLKHGADVNAQDKFGKTPFDLAIDNGNE
DIAEVLQKAA
SEQ ID NO: 2
GACCTGGGTAAGAAACTGCTGGAAGCTGCTCGTGCTGGTCAGGACGACG
AAGTTCGTATCCTGATGGCAAACGGTGCTGACGTTAACGCTAACGACCG
TAACGGTGTTACTCCGCTGCACCTGGCTGCTGACAAAGGTCACCTGGAA
ATCGTTGAAGTTCTGCTGAAAACCGGTGCTGACGTTAACGCTATCGACA
TCATGGGTGCTACTCCGCTGCACCTGGCTGCTGCTCATGGTCACCTGGA
AATCGTTGAAGTTCTGCTGAAGGCCGGCGCTGACGTTAACGCTATGGAC
CATAAAGGTTTCACTCCGCTGCACCTGGCTGCTTGGCGTGGTCACCTGG
AAATCGTTGAAGTTCTGCTGAAGCACGGCGCCGACGTTAACGCTCAGGA
CAAATTCGGTAAGACTCCGTTCGACCTGGCTATCGACAACGGTAACGAG
GACATCGCTGAAGTTCTGCAGAAAGCTGCT
SEQ ID NO: 3
MRGSHHHHHHHHGSDLGKKLLEAARAGQDDEVRILMANGADVNANDRNG
VTPLHLAADKGHLEIVEVLLKTGADVNAIDIMGATPLHLAAAHGHLEIV
EVLLKAGADVNAMDHKGFTPLHLAAWRGHLEIVEVLLKHGADVNAQDKF
GKTPFDLAIDNGNEDIAEVLQKAAKLNDYKDDDDK
SEQ ID NO: 4
ATGAGAGGATCGCATCACCATCACCATCACCATCACGGATCCGACCTGG
GTAAGAAACTGCTGGAAGCTGCTCGTGCTGGTCAGGACGACGAAGTTCG
TATCCTGATGGCAAACGGTGCTGACGTTAACGCTAACGACCGTAACGGT
GTTACTCCGCTGCACCTGGCTGCTGACAAAGGTCACCTGGAAATCGTTG
AAGTTCTGCTGAAAACCGGTGCTGACGTTAACGCTATCGACATCATGGG
TGCTACTCCGCTGCACCTGGCTGCTGCTCATGGTCACCTGGAAATCGTT
GAAGTTCTGCTGAAGGCCGGCGCTGACGTTAACGCTATGGACCATAAAG
GTTTCACTCCGCTGCACCTGGCTGCTTGGCGTGGTCACCTGGAAATCGT
TGAAGTTCTGCTGAAGCACGGCGCCGACGTTAACGCTCAGGACAAATTC
GGTAAGACTCCGTTCGACCTGGCTATCGACAACGGTAACGAGGACATCG
CTGAAGTTCTGCAGAAAGCTGCTAAGCTTAATGACTACAAGGATGACGA
CGACAAG

### DESCRIPTION OF THE FIGURES

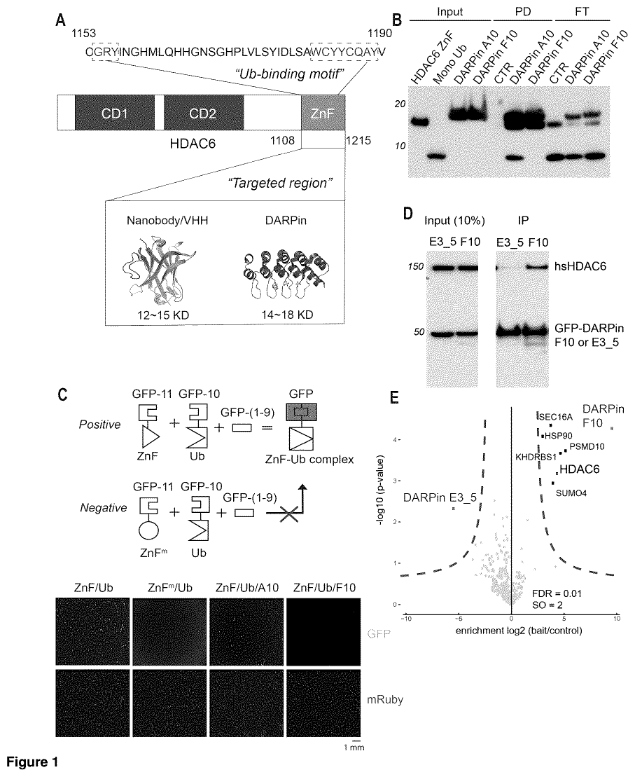

FIG. 1: DARPin F10 inhibits with high specificity ZnF and Ub interaction in vitro and in vivo.
(A) Schematic of HDAC6, showing the catalytic domains (CD1, CD2) and the zinc finger domain (ZnF) region that was expressed (amino acids 1108 to 1215) and used to identify binders. Generic nanobody and DARPin structures (PDB:1I3V and PDB:2QJY, respectively) are shown below. Part of the HDAC6 sequence (1153 to 1190) is indicated at top to present the ubiquitin binding motifs (Uniprot: Q9UBN7)(Ouyang et al., 2012), which are coloured in red and framed.
(B) DARPin F10 blocks Ub and ZnF domain interaction in vitro. Purified His-tagged ZnF domain (1108-1215), Flag-tagged DARPin A10 or F10 and mono-ubiquitin were mixed together for a binding reaction; following incubation, the ZnF domain was pulled down with anti-Flag agarose beads. The precipitated complex was eluted and analysed by immunoblotting, using anti-His, anti-ubiquitin and anti-Flag antibodies. PD, pull-down; FT, flow-through.
(C) In vivo interaction between the ZnF domain and Ub is disrupted by DARPin F10, as monitored by a split-GFP assay. The ZnF domain (1108-1215) and Ub were fused to separate GFP fragments so that ZnF-Ub interaction is required to reconstitute a functional GFP molecule and fluorescence (see scheme at top). The GFP beta strands encoded in the different proteins are indicated: GFP-(1-9), GFP-10, GFP-11. A mutant ZnF domain (W1182A; ZnF^{m}) which cannot interact with Ub was also used as a control for specificity of the assay. A plasmid expressing mRuby was included in all transfections as a control for transfection efficiency (red signal). Scale bar presents 1 mm.
(D) Efficient immunoprecipitation of endogenous HDAC6 by DARPin F10. A GFP-DARPin F10 or control DARPin E3_5 fusion protein was transiently expressed in A549 cells, and the DARPins were immunoprecipitated with GFP-trap beads. The immunoprecipitated material (IP) was eluted and analysis was done by immunoblotting, using antibodies against GFP or HDAC6.
(E) Mass spectrometry analysis to determine the interactome of DARPin F10. Immunoprecipitated material (from D above) was analysed by mass spectrometry and enriched proteins are annotated.

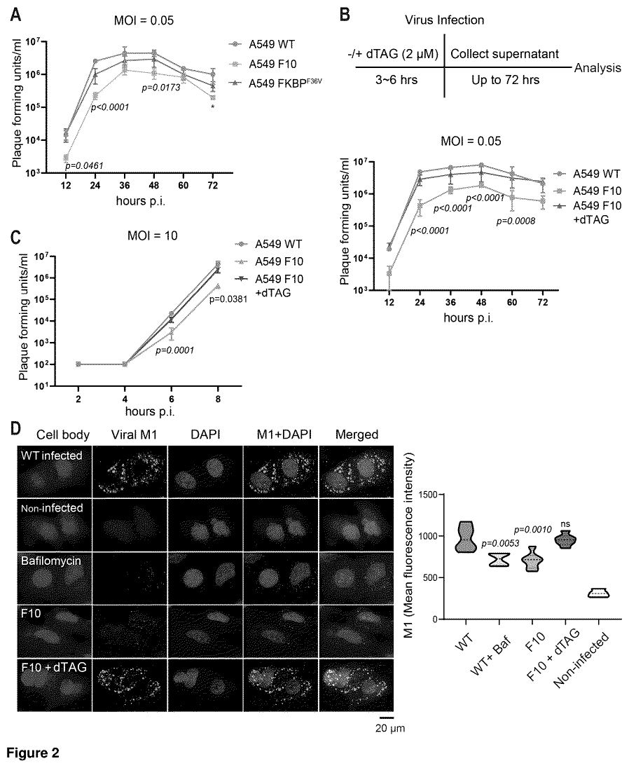

FIG. 2: Blocking the HDAC6 ZnF-Ub interaction by DARPin F10 interferes with IAV infection
(A) DARPin F10 impairs IAV infection. The indicated cell lines (A549 WT, F10 or FKBP^{F36V}) were infected with IAV at a multiplicity of infection (MOI) of 0.05 (n=3), and culture supernatants were collected every 12 hrs until 72 hrs. Viral titer in supernatant was quantified by plaque assay. Statistical analysis was done with ANOVA, and p-value is shown in the graph to illustrate the significant difference (FDR<0.05) between cell lines expressing or not DARPin F10.
(B) Treatment with dTAG restores IAV susceptibility. The indicated cell lines (A549 WT, DARPin F10 without or with dTAG pre-treatment) were infected with IAV at a MOI of 0.05 (n=3), and culture supernatants were collected every 12 hrs until 72 hrs. Viral titer in supernatant was quantified by plaque assay. Statistical analysis was done with ANOVA, and p-value is shown in the graph to illustrate the significant difference (FDR<0.05) between A549 WT and F10 cell line.
(C) Effect of DARPin F10 on a single IAV life cycle. The indicated cell lines were infected with IAV at a MOI of 10 (n=3) and culture supernatants were collected every 2 hrs up to 8 hrs. Viral titer was determined by plaque assay. Statistical analysis was done with ANOVA test, and p-value is shown in the graph to illustrate the significant difference (FDR<0.05) between A549 WT and F10 cell line.
(D) IAV uncoating is impaired by DARPin F10. The left panels show confocal microscopy visualization of uncoating, by staining for the viral capsid M1 protein (green). Parental A549 cells or F10 cells (without or with dTAG pre-treatment) were used for IAV infection and M1 expression was analysed 3.5 hrs post infection. Bafilomycin A1 treatment was used as a control for blocked infection. Total protein was stained to visualize the cell body (red). The right panel presents a quantification of the M1 analysis in the different samples. Ca. 30 cells were selected per view (6 to 9 views for each condition) and M1 fluorescence intensity was analysed. The p-value, indicating the difference against A549 WT, was calculated by ANOVA test (with a FDR<0.05). The scale bar represents 20 μm.

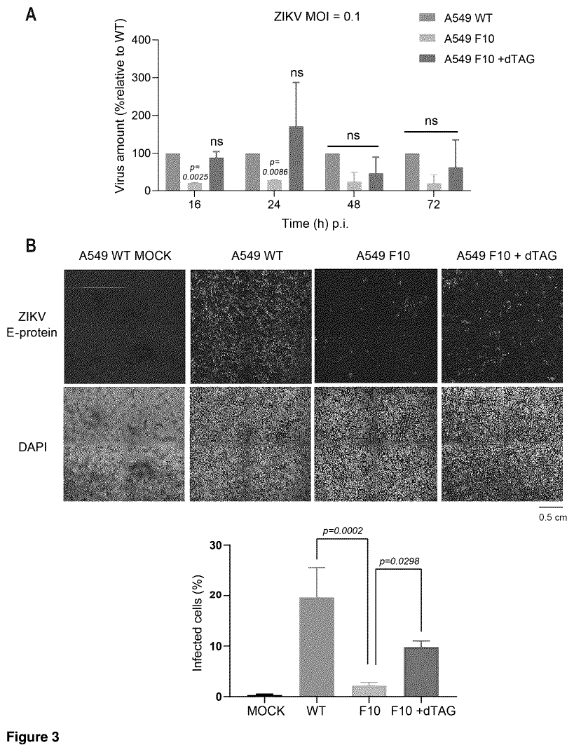

FIG. 3: ZIKV replication is inhibited by DARPin F10.
(A) Reduction of ZIKV titer in DARPin F10-expressing cells. The indicated cell lines (WT, DARPin F10 without or with dTAG pre-treatment) were infected with ZIKV at a MOI of 0.1 fifty-percent tissue culture infective dose (TCID₅₀)/cell and culture supernatants were collected at 16, 24, 48 and 72 hrs post-infection. Viral titers were determined with a TCID₅₀ assay; the baseline titer obtained with A549 WT cells at each time point was set to 100%. Statistical analysis was done with ANOVA, and p-value is shown in the graph to illustrate the significant difference (FDR<0.05) between A549 WT and each cell line; ns, non-significant.
(B) Reduced ZIKV infection in the F10 cell line. The upper panels show microscopy visualization of infection. Following ZIKV titer quantification in (A), the cells at 72 hrs post-infection were stained by DAPI and for ZIKV E protein. A combination of the neighbouring 4 views (10× objective, containing 6500 to 7000 cells) showed a strong reduction in the number of cells positive for ZIKV E protein when F10 was expressed. The lower panels present a quantification (n=3) of the ZIKV E protein-positive cells. Statistical analysis was done with ANOVA; p-values refer to the significant difference (FDR<0.05) between samples, as indicated. The scale bar represents 0.5 cm.

### DETAILED DESCRIPTION OF THE INVENTION

Previously, some of the present inventors found that a therapeutically effective amount of a modulator of the ubiquitin-binding property of HDAC6 could be used to treat virus infections. While trying to generate designed ankyrin repeat proteins (DARPins) and nanobodies inhibiting the ubiquitin-binding property of HDAC6, the inventors realised that the search for such agents is not straightforward. The inventors used purified human HDAC6 ZnF domain to identify DARPINS and nanobodies binding specifically to this domain. Among the numerous binding molecules identified as binding specifically to human HDAC6 ZnF domain, only one single DARPin, called F10 and having the amino acid sequence of SEQ ID NO:1, was identified as having the desired biological property of blocking the ubiquitin-engaging zinc finger domain of HDAC6.

The present invention hence provides recombinant binding protein comprising at least 90 consecutive amino acids of SEQ ID NO:1, wherein said recombinant binding protein specifically binds to HDAC6 and blocks the ubiquitin-engaging zinc finger domain of HDAC6. In one embodiment, the recombinant binding protein of the invention comprises an ankyrin repeat domain corresponding to amino acids 25 to 129 of SEQ ID NO:1. In another embodiment, the recombinant binding protein of the invention comprises an ankyrin repeat domain corresponding to amino acids 31 to 123 of SEQ ID NO:1. The recombinant binding protein of the invention can comprises 90, 95, 100, 105, 110, 115, 120, 125, 130,135, 140, 145, 150, 155, or 157 consecutive amino acids of SEQ ID NO:1, In one embodiment, the recombinant binding protein of the invention comprises at least 120 consecutive amino acids of SEQ ID NO:1. In one embodiment, the recombinant binding protein of the invention comprises an ankyrin repeat domain corresponding to amino acids 1 to 129 of SEQ ID NO:1. In another embodiment, the recombinant binding protein of the invention comprises an ankyrin repeat domain corresponding to amino acids 1 to 123 of SEQ ID NO:1. In one embodiment, the recombinant binding protein of the invention comprises an ankyrin repeat domain corresponding to amino acids 25 to 157 of SEQ ID NO:1. In another embodiment, the recombinant binding protein of the invention comprises an ankyrin repeat domain corresponding to amino acids 31 to 157 of SEQ ID NO:1. In one embodiment, the recombinant binding protein of the invention comprises SEQ ID NO:1. The present invention also provides a recombinant binding protein that competes for binding to HDAC6 with the recombinant binding protein described in this paragraph and blocks the ubiquitin-engaging zinc finger domain of HDAC6. The term “compete for binding” means the inability of two different recombinant binding proteins to bind simultaneously to the same target, while both are able to bind to this target individually, and can be routinely assessed by the practitioner in the art.

In some embodiments, the recombinant binding protein of the invention binds to HDAC6 with a K_{D} below 4 μM. As well known to the skilled person, the equilibrium dissociation constant (K_{D}) is the basic parameter to evaluate the binding property of the drug-receptor and can be determined by, a variety of analytical methods, including radioligand binding assay, surface plasmon resonance method, fluorescence energy resonance transfer method, affinity chromatography, and isothermal titration calorimetry.

The present invention also provides an isolated nucleic acid encoding a recombinant binding protein according to any of the preceding claims. In one embodiment, this isolated nucleic acid of the invention comprises the nucleic acid sequence of SEQ ID NO:2. For the example, the isolated nucleic acid molecule of the invention can have the nucleic acid sequence of SEQ ID NO:4, and code for the recombinant binding protein having the amino acid sequence of SEQ ID NO:3 (F10 with histidine and detection tags attached to it by BamH1 and HindIII sites, respectively).

The isolated nucleic acid of the invention can be integrated within an expression cassette and operatively linked to genetic elements, e.g. promoter, operator, regulator, or transient repressor, allowing and/or controlling its expression in target cells. Genetic elements allowing and/or controlling expression in target cells are well known to the skilled person. In some embodiments, the elements controlling the expression of the isolated nucleic acid allow for conditional expression. In addition, elements that will lead to the fusion of a cell-penetrating peptide, such as the HIV TAT protein, to the recombinant binding protein of the invention can also be used. The cell-permeable peptide can be fused to the DARPin at its C-terminus or at its N-terminus.

The present invention further comprises vectors comprising the expression cassette and isolated nucleic acids of the invention. In some embodiments, the vector can be a mRNA molecule. In some embodiments, this vector can be a viral vector, for instance the vector can be an AAV, a PRV or a lentivirus. In some embodiments, it is an AAV.

The recombinant binding proteins, isolated nucleic acids, expression cassettes or vectors of the invention can be used as medicament, for instance to treat viral infections. The present invention also provides pharmaceutical compositions comprising them, and optionally a pharmaceutical acceptable carrier and/or diluent.

The present invention provides methods for treating a viral infection in a subject characterised in that a therapeutically effective amount of recombinant binding proteins, isolated nucleic acids, expression cassettes, vectors or pharmaceutical compositions of the invention is administered to said subject. In some embodiments, the virus is an enveloped virus, for instance an influenza virus, a zika virus or an ebola virus.

The sequences of the invention are:

SEQ ID NO: 1
DLGKKLLEAARAGQDDEVRILMANGADVNANDRNGVTPLHLAADKGHLE
IVEVLLKTGADVNAIDIMGATPLHLAAAHGHLEIVEVLLKAGADVNAMD
HKGFTPLHLAAWRGHLEIVEVLLKHGADVNAQDKFGKTPFDLAIDNGNE
DIAEVLQKAA
SEQ ID NO: 2
GACCTGGGTAAGAAACTGCTGGAAGCTGCTCGTGCTGGTCAGGACGACG
AAGTTCGTATCCTGATGGCAAACGGTGCTGACGTTAACGCTAACGACCG
TAACGGTGTTACTCCGCTGCACCTGGCTGCTGACAAAGGTCACCTGGAA
ATCGTTGAAGTTCTGCTGAAAACCGGTGCTGACGTTAACGCTATCGACA
TCATGGGTGCTACTCCGCTGCACCTGGCTGCTGCTCATGGTCACCTGGA
AATCGTTGAAGTTCTGCTGAAGGCCGGCGCTGACGTTAACGCTATGGAC
CATAAAGGTTTCACTCCGCTGCACCTGGCTGCTTGGCGTGGTCACCTGG
AAATCGTTGAAGTTCTGCTGAAGCACGGCGCCGACGTTAACGCTCAGGA
CAAATTCGGTAAGACTCCGTTCGACCTGGCTATCGACAACGGTAACGAG
GACATCGCTGAAGTTCTGCAGAAAGCTGCT
SEQ ID NO: 3
MRGSHHHHHHHHGSDLGKKLLEAARAGQDDEVRILMANGADVNANDRNG
VTPLHLAADKGHLEIVEVLLKTGADVNAIDIMGATPLHLAAAHGHLEIV
EVLLKAGADVNAMDHKGFTPLHLAAWRGHLEIVEVLLKHGADVNAQDKF
GKTPFDLAIDNGNEDIAEVLQKAAKLNDYKDDDDK
SEQ ID NO: 4
ATGAGAGGATCGCATCACCATCACCATCACCATCACGGATCCGACCTGG
GTAAGAAACTGCTGGAAGCTGCTCGTGCTGGTCAGGACGACGAAGTTCG
TATCCTGATGGCAAACGGTGCTGACGTTAACGCTAACGACCGTAACGGT
GTTACTCCGCTGCACCTGGCTGCTGACAAAGGTCACCTGGAAATCGTTG
AAGTTCTGCTGAAAACCGGTGCTGACGTTAACGCTATCGACATCATGGG
TGCTACTCCGCTGCACCTGGCTGCTGCTCATGGTCACCTGGAAATCGTT
GAAGTTCTGCTGAAGGCCGGCGCTGACGTTAACGCTATGGACCATAAAG
GTTTCACTCCGCTGCACCTGGCTGCTTGGCGTGGTCACCTGGAAATCGT
TGAAGTTCTGCTGAAGCACGGCGCCGACGTTAACGCTCAGGACAAATTC
GGTAAGACTCCGTTCGACCTGGCTATCGACAACGGTAACGAGGACATCG
CTGAAGTTCTGCAGAAAGCTGCTAAGCTTAATGACTACAAGGATGACGA
CGACAAG

The term “protein” refers to a polypeptide, wherein at least part of the polypeptide has, or is able to acquire a defined three-dimensional arrangement by forming secondary, tertiary, or quaternary structures within and/or between its polypeptide chain(s). If a protein comprises two or more polypeptides, the individual polypeptide chains may be linked non-covalently or covalently, e.g. by a disulfide bond between two polypeptides. A part of a protein, which individually has, or is able to acquire a defined three-dimensional arrangement by forming secondary or tertiary structures, is termed “protein domain”. Such protein domains are well known to the practitioner skilled in the art.

The term “recombinant” as used in recombinant protein, recombinant protein domain and the like, means that said polypeptides are produced by the use of recombinant DNA technologies well known by the practitioner skilled in the relevant art. For example, a recombinant DNA molecule (e.g. produced by gene synthesis) encoding a polypeptide can be cloned into a bacterial expression plasmid (e.g. pQE30, Qiagen). When such a constructed recombinant expression plasmid is inserted into a bacteria (e.g. E. coli), this bacteria can produce the polypeptide encoded by this recombinant DNA. The correspondingly produced polypeptide is called a recombinant polypeptide.

The term “polypeptide tag” refers to an amino acid sequence attached to a polypeptide/protein, wherein said amino acid sequence is useful for the purification, detection, or targeting of said polypeptide/protein, or wherein said amino acid sequence improves the physicochemical behavior of the polypeptide/protein, or wherein said amino acid sequence possesses an effector function. The individual polypeptide tags, moieties and/or domains of a binding protein may be connected to each other directly or via polypeptide linkers. These polypeptide tags are all well known in the art and are fully available to the person skilled in the art. Examples of polypeptide tags are small polypeptide sequences, for example, His, myc, FLAG, or Strep-tags or moieties such as enzymes (for example enzymes like alkaline phosphatase), which allow the detection of said polypeptide/protein, or moieties which can be used for targeting (such as immunoglobulins or fragments thereof) and/or as effector molecules.

The term “polypeptide linker” refers to an amino acid sequence, which is able to link, for example, two protein domains, a polypeptide tag and a protein domain, a protein domain and a non-polypeptide moiety such as polyethylene glycol or two sequence tags. Such additional domains, tags, non-polypeptide moieties and linkers are known to the person skilled in the relevant art. A list of example is provided in the description of the patent application WO 02/20565. Particular examples of such linkers are glycine-serine-linkers and proline-threonine-linkers of variable lengths; preferably, said linkers have a length between 2 and 24 amino acids; more preferably, said linkers have a length between 2 and 16 amino acids.

In the context of the present invention, the term “polypeptide” relates to a molecule consisting of one or more chains of multiple, i.e. two or more, amino acids linked via peptide bonds. Preferably, a polypeptide consists of more than eight amino acids linked via peptide bonds.

The term “polymer moiety” refers to either a proteinaceous polymer moiety or a non-proteinaceous polymer moiety. A “proteinaceous polymer moiety” preferably is a polypeptide that does not form a stable tertiary structure while not forming more than 10% (preferably, not more than 5%; also preferred, not more than 2%; even more preferably, not more than 1%; and most preferably, no detectable amounts, as determined by size exclusion chromatography (SEC)) of oligomers or aggregates when stored at a concentration of about 0.1 mM in PBS at RT for one month. Such proteinaceous polymer moieties run at an apparent molecular weight in SEC that is higher than their effective molecular weight when using globular proteins as molecular weight standards for the SEC. Preferably, the apparent molecular weight of said proteinaceous polymer moieties determined by SEC is 1.5×, 2× or 2.5× higher than their effective molecular weight calculated from their amino acid sequence. Also preferably, the apparent molecular weights of said non-proteinaceous polymer moieties determined by SEC is 2×, 4× or 8× higher than their effective molecular weight calculated from their molecular composition. Preferably, more than 50%, 70% or even 90% of the amino acids of said proteinaceous polymer moiety do not form stable secondary structures at a concentration of about 0.1 mM in PBS at RT as determined by Circular Dichroism (CD) measurements. Most preferably, said proteinaceous polymer shows a typical near UV CD-spectra of a random coil conformation. Such CD analyses are well known to the person skilled in the art. Also preferable are proteinaceous polymer moieties that consist of more than 50, 100, 200, 300, 400, 500, 600, 700 or 800 amino acids. Examples of proteinaceous polymer moieties are XTEN® (a registered trademark of Amunix; WO 07/103515) polypeptides, or polypeptides comprising proline, alanine and serine residues as described in WO 08/155134.

Examples of non-proteinaceous polymer moieties are hydroxyethyl starch (HES), polyethylene glycol (PEG), polypropylene glycol, or polyoxyalkylene. The term “PEGylated” means that a PEG moiety is covalently attached to, for example, a polypeptide of the invention.

In a specific embodiment, a PEG moiety or any other non-proteinaceous polymer can, e.g., be coupled to a cysteine thiol via a maleimide linker with the cysteine being coupled via a peptide linker to the N- or C-terminus of a binding domain as described herein.

The term “binding protein” refers to a protein comprising one or more binding domains and one or more polymer moieties as further explained below. Said binding protein can comprise up to four binding domains. Said binding protein can comprise up to two binding domains. Said binding protein can comprise only one binding domain. Furthermore, any such binding protein may comprise additional protein domains that are not binding domains, multimerization moieties, polypeptide tags, polypeptide linkers and/or a single Cys residue. Examples of multimerization moieties are immunoglobulin heavy chain constant regions which pair to provide functional immunoglobulin Fc domains, and leucine zippers or polypeptides comprising a free thiol which forms an intermolecular disulfide bond between two such polypeptides. The single Cys residue may be used for conjugating other moieties to the polypeptide, for example, by using the maleimide chemistry well known to the person skilled in the art.

Also preferably, said binding protein has an apparent molecular weight of at least 70, 100, 200, 300, 500 or 800 kDa when analyzed at a concentration of 0.1 mM in PBS at RT by SEC using globular proteins as molecular weight standards.

The term “binding domain” means a protein domain exhibiting the same “fold” (three-dimensional arrangement) as a protein scaffold and having a predetermined property, as defined below. Such a binding domain may be obtained by rational, or most commonly, combinatorial protein engineering techniques, skills which are known in the art (Skerra, 2000, loc. cit; Binz et al., 2005, loc. cit). For example, a binding domain having a predetermined property can be obtained by a method comprising the steps of (a) providing a diverse collection of protein domains exhibiting the same fold as a protein scaffold as defined further below; and (b) screening said diverse collection and/or selecting from said diverse collection to obtain at least one protein domain having said predetermined property. The diverse collection of protein domains may be provided by several methods in accordance with the screening and/or selection system being used, and may comprise the use of methods well known to the person skilled in the art, such as phage display or ribosome display.

The term “protein scaffold” means a protein with exposed surface areas in which amino acid insertions, substitutions or deletions are highly tolerable. Examples of protein scaffolds that can be used to generate binding domains of the present invention are antibodies or fragments thereof such as single-chain Fv or Fab fragments, protein A from Staphylococcus aureus, the bilin binding protein from Pieris brassicae or other lipocalins, ankyrin repeat proteins or other repeat proteins, and human fibronectin. Protein scaffolds are known to the person skilled in the art (Binz et al., 2005, loc. cit.; Binz et al., 2004, loc. cit.).

The term “predetermined property” refers to a property such as binding to a target, blocking of a target, activation of a target-mediated reaction, enzymatic activity, and related further properties. Depending on the type of desired property, one of ordinary skill will be able to identify format and necessary steps for performing screening and/or selection of a binding domain with the desired property. Preferably, said predetermined property is binding to a target.

The term “capping module” refers to a polypeptide fused to the N- or C-terminal repeat module of a repeat domain, wherein said capping module forms tight tertiary interactions with said repeat module thereby providing a cap that shields the hydrophobic core of said repeat module at the side not in contact with the consecutive repeat module from the solvent. Said N- and/or C-terminal capping module may be, or may be derived from, a capping unit or other domain found in a naturally occurring repeat protein adjacent to a repeat unit. The term “capping unit” refers to a naturally occurring folded polypeptide, wherein said polypeptide defines a particular structural unit which is N- or C-terminally fused to a repeat unit, wherein said polypeptide forms tight tertiary interactions with said repeat unit thereby providing a cap that shields the hydrophobic core of said repeat unit at one side from the solvent. Such capping units may have sequence similarities to said repeat sequence motif. Capping modules and capping repeats are described in WO 02/020565.

As used herein, the term “population” may be any group of at least two individuals. A population may include, e.g., but is not limited to, a reference population, a population group, a family population, a clinical population, and a same sex population.

As used herein, the term “polymorphism” means any sequence variant present at a frequency of >1% in a population. The sequence variant may be present at a frequency significantly greater than 1% such as 5% or 10% or more. Also, the term may be used to refer to the sequence variation observed in an individual at a polymorphic site. Polymorphisms include nucleotide substitutions, insertions, deletions and microsatellites and may, but need not, result in detectable differences in gene expression or protein function.

As used herein, the term “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified e.g. for stability or for other reasons.

As used herein, the term “reference standard population” means a population characterized by one or more biological characteristics, e.g., drug responsiveness, genotype, haplotype, phenotype, etc.

As used herein, the term “subject” means that preferably the subject is a mammal, such as a human, but can also be an animal, including but not limited to, domestic animals (e.g., dogs, cats and the like), farm animals (e.g., cows, sheep, pigs, horses and the like) and laboratory animals (e.g., monkeys such as cynomolgous monkeys, rats, mice, guinea pigs and the like).

As used herein, the term “statistically significant” means a p value <0.05 as compared to the control using the Student's t-test.

The present invention is also directed to therapies which involve administering the reagents of the invention, in some embodiments, a mammal, for example a human, patient to treat virus infections. The subject is in some embodiments, an animal, including but not limited to animals such as cows, pigs, horses, chickens, cats, dogs, etc., and is in some embodiments, a mammal, for example human.

Various delivery systems are known and can be used to administer a therapeutic agent, e.g., encapsulation in liposomes, microparticles, microcapsules, recombinant cells capable of expressing the compound, receptor-mediated endocytosis (see, e. g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the agent or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref, Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g. Goodson, in Medical Applications of Controlled Release, supra, vol. 2, pp. 115-13 8 (1984)). Other controlled release systems are discussed in the review by Langer (Science 249:1527-1533 (1990)).

The present invention also provides pharmaceutical compositions for use in the treatment of enveloped viruses, such as influenza. Such compositions comprise a therapeutically effective amount of an inhibitory compound, and a pharmaceutically acceptable carrier. In a specific embodiment, the term “pharmaceutically acceptable” means approved by a regulatory agency of the Federal or a state government or listed in the U. S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection.

Generally, the ingredients are supplied either separately or mixed together in unit dosage form, for example, as a dry lyophilized powder or water free concentrate in a hermetically scaled container such as an ampoule or sachette indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances.

Effective doses may be extrapolated from dose-response curves derived from in vitro or animal model test systems. For antibodies, the dosage administered to a patient is typically 0.1 mg/kg to 100 mg/kg of the patient's body weight. In some embodiments, the dosage administered to a patient is between 0.1 mg/kg and 20 mg/kg of the patient's body weight, for example 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Also encompassed is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The inhibitors of the invention may also be incorporated within a slow or delayed release device. Such devices may, for example, be inserted in the body of the subject, and the molecule may be released over weeks or months. Such devices may be particularly advantageous when long term treatment with an antagonist of HDAC6 is required and which would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the subject being treated.

Optimal dosages to be administered may be determined by those skilled in the art, and will vary with the particular inhibitor in use, the strength of the preparation, and the mode of administration. Additional factors depending on the particular subject being treated will result in a need to adjust dosages, including subject age, weight, gender, diet, and time of administration.

When the inhibitor is a nucleic acid conventional molecular biology techniques (vector transfer, liposome transfer, ballistic bombardment etc) may be used to deliver the inhibitor to the target tissue. Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Cell-penetrating peptides (CPPs) are short peptides that facilitate cellular intake and uptake of molecules ranging from nanosize particles to small chemical compounds to large fragments of DNA. The “cargo” is associated with the peptides either through chemical linkage via covalent bonds or through non-covalent interactions. CPPs deliver the cargo into cells commonly through endocytosis. CPPs typically have an amino acid composition that either contains a high relative abundance of positively charged amino acids such as lysine or arginine or has sequences that contain an alternating pattern of polar, charged amino acids and non-polar, hydrophobic amino acids. These two types of structures are referred to as polycationic or amphipathic, respectively. A third class of CPPs are the hydrophobic peptides, containing only apolar residues with low net charge or hydrophobic amino acid groups that are crucial for cellular uptake. Transactivating transcriptional activator (TAT), from human immunodeficiency virus 1 (HIV-1), was the first CPP discovered. Since then, the number of known CPPs has expanded considerably, and small molecule synthetic analogues with more effective protein transduction properties have been generated.

HDAC6, also known as histone deacetylase 6, EC 3.5.1.983, HD6, JM211, FLJ16239, OTTHUMP00000032398, KIAA0901, or OTTHUMP00000197663, plays a central role in microtubule-dependent cell motility via deacetylation of tubulin. In addition to its protein deacetylase activity, HDAC6 binds with high affinity ubiquitin or ubiquitinated proteins and plays a key role in the management of misfolded proteins, i.e. when misfolded proteins are too abundant to be degraded by the chaperone refolding system and the ubiquitin-proteasome system, HDAC6 mediates the transport of misfolded proteins to the aggresome, a cytoplasmic juxtanuclear structure and also promotes the formation of stress granules. HDAC6 belongs to class IIb of the histone deacetylase/acuc/apha family. It contains an internal duplication of two catalytic domains which appear to function independently of each other. This protein possesses histone deacetylase activity and can repress transcription if present in the nucleus. Additional known substrates of HDAC6 are the chaperone Hsp90 or the actin-binding protein cortactin. In some experiments HDAC6 has been shown to deacetylate the N-terminal tails of histones. Histone deacetylation gives a tag for epigenetic repression and plays an important role in transcriptional regulation, cell cycle progression and developmental events.

The HDAC6 gene is expressed relatively ubiquitously and is not known to be induced in response to stimuli. It has been shown that acetylation of HDAC6 by p300 attenuates its deacetylase activity (Han Y et al., 2009). Also, Aurora kinase A (AurA) colocalizes with HDAC6 at the basal body of cilia and phosphorylates it, thereby enhancing its tubulin deacetylase activity (Pugacheva et al., 2007). Furthermore, it was also shown that protein kinase CKII phosphorylates HDAC6 on Serine 458, increasing its deacetylase activity and promoting formation and clearance of aggresomes (Watabe and Nakaki, 2012).

As used herein, the “enzymatic activity of HDAC6” refers to the enzymatic (deacetylase) activity of HDAC6, whereas the capacity of HDAC6 to bind ubiquitinated proteins is referred to as “ubiquitin-binding activity of HDAC6” or “ubiquitin-binding property of HDAC6”.

In the context of the present invention, i.e. for the treatment of viral infections, the terms “antagonist of HDAC6” or “inhibitors of HDAC6” refers to agents/molecules which specifically block or strongly reduce the ubiquitin-binding activity of HDAC6.

Influenza, commonly known as “the flu”, is an infectious disease of birds and mammals caused by RNA viruses of the family Orthomyxoviridae, the influenza viruses.

The Orthomyxoviruses (orthos, Greek for “straight”; myxa, Greek for “mucus”) are a family of RNA viruses that includes six genera: Influenza virus A, Influenza virus B, Influenza virus C, Isavirus, Thogotovirus and a recently discovered, still undescribed genus. The first three genera contain viruses that cause influenza in vertebrates, including birds (see also avian influenza), humans, and other mammals. Isaviruses infect salmon; thogotoviruses infect vertebrates and invertebrates, such as mosquitoes and sea lice.

The Influenza viruses A, B and C, which are identified by antigenic differences in their nucleoprotein and matrix protein, infect vertebrates as follows: Influenza virus A infects humans, other mammals, and birds, and causes all flu pandemics; Influenza virus B infects humans and seals; Influenza virus C infects humans and pigs.

“Enveloped viruses” are viruses having a viral envelope. A category of enveloped viruses is enveloped RNA viruses. A viral envelope is the outermost layer of many types of viruses. It protects the genetic material in their life-cycle when traveling between host cells. The envelopes are typically derived from portions of the host cell membranes (phospholipids and proteins), but include some viral glycoproteins.

All enveloped viruses also have a capsid, another protein layer, between the envelope and the genome. Enveloped viruses possess great adaptability and can change in a short time in order to evade the immune system. Enveloped viruses can cause persistent infections. Examples of enveloped viruses are, Herpesviruses, Poxviruses, Hepadnaviruses, Asfarviridae, Flavivirus, Alphavirus, Togavirus, Coronavirus, Hepatitis D, Orthomyxovirus, Paramyxovirus, Rhabdovirus, Bunyavirus, Filovirus and Retroviruses

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Examples

### Nanobody Screens & Preparation

The target protein was a 6×His- and HALO-tagged human HDAC6 ZnF domain (aa 1106-1215, expressed from plasmid pHis6HaloTag-hHDAC6ZnF) which was prepared by expression in E. coli BL21(DE3)RIL₊. As a control, 6×His-HALO protein (expressed from plasmid pH6HTN His6HaloTag) was prepared in a similar manner. Cells were induced with 0.5 mM IPTG at 20° C. for 20 h. E. coli BL21 (DE3) cells expressing 6×His-HALO-tagged ZnF—UBP were pelleted, rapidly frozen in liquid nitrogen and stored at −80° C. The frozen cells were resuspended in ice-cold lysis buffer (20 mM Tris, pH7.5, 200 mM NaCl, 20 mM imidazole, 2 mM TCEP, 0.2% Tween-20) supplemented with Complete EDTA-free protease inhibitors (Roche) and 3 U/ml Benzonase (Sigma). After 30 min on ice the lysate was centrifuged at 30,000 g for 30 min at 4° C. The clarified soluble lysate was incubated for 30 min at 4° C. in batch mode with Ni-NTA IMAC agarose (Qiagen), and then transferred into a 10 ml Econo-Pac column (Bio-Rad) for washing with nickel wash buffer (20 mM Tris, pH 7.5, 200 mM NaCl, 20 mM imidazole, 2 mM TCEP). The target protein was eluted in nickel wash buffer containing 250 mM imidazole. The eluted protein was concentrated with Amicon ultra concentration device (30,000 MWCO)(Millipore) and separated using a DUO FLOW system (Bio-Rad) with a Sephacryl S-300 16/60 gel filtration column (GE Healthcare) equilibrated in 20 mM Tris, pH 7.5, 200 mM NaCl, 2 mM TCEP, 5% Glycerol and 0.02% NaN3. Protein fractions were analyzed on a 4-12% Bis-Tris NuPAGE (Invitrogen) gels and pure fractions were pooled and concentrated to 15 mM for Nanobody production. Gels were stained with InstantBlue (Expedeon). Identification of nanobodies against the HDAC6 ZnF domain was done by Hybrigenics Services SAS. In brief, HALO-ZnF (or HALO as control) protein was biotinylated in vitro using HaloTag® PEG-Biotin Ligand (Promega: G8591 or G8592) following the manufacturer's instruction and then used for three rounds of phage display with a naïve synthetic library based on a proprietary Lama scaffold. The Phage library was first incubated with the biotinylated His-HALO; the supernatant was then incubated with the biotinylated HALO-ZnF. Following selection, the positive hits were used to generate a yeast two-hybrid library, which was then screened against the human HDAC ZnF domain as bait (aa 1106-1215). Positive hits were isolated and validated by an intrabody assay. Following this, four different positive clones (Nb1 to 4) as well as a control clone were selected for further analysis.

GFP-Trap Pull-Down Assay

C-terminally eGFP-tagged nanobodies were cloned into pLVX-puro lentiviral expression vectors. The lentiviral vector was co-transfected with Pol-Gag and VSV-G plasmids into HEK293T cells to produce lentivirus. Each eGFP-tagged nanobody was stably expressed in A549 cells after lentivirus infection, then eGFP-positive cells were sorted by FACS. A549 cells expressing each nanobody were harvested with ice-cold PBS from a 10 cm dish, spun down at 1,000 g for 5 min. The pelleted samples were rapidly frozen at −80° C. The frozen pellet was treated with CSK (cytoskeleton) buffer (10 mM PIPES pH6.8, 300 mM sucrose, 100 mM NaCl, 3 mM MgCl₂, 1 mM EGTA, 0.1% (v/v) Triton X-100) with 1× Complete EDTA free protease inhibitor cocktail (Roche #CO-RO) for 30 min on ice. The lysed cell extracts were subjected to low-speed centrifugation (3,000 rpm for 5 min) to separate the soluble cytoplasmic fraction. eGFP-tagged nanobodies were pulled-down with GFP-Trap agarose beads (Chromotek #gtm-20) equilibrated with CSK buffer containing 1% BSA. The soluble fractions were incubated with GFP-Trap beads for 30 min at 4° C., then spun down 1,000 g for 2 min. The beads were washed with GFP-Trap Wash buffer (10 mM Tris, pH7.5, 200 mM NaCl, 0.5 mM EDTA, 5% Glycerol) twice, and finally washed with 10 mM Tris, pH7.5, 100 mM NaCl, 5% Glycerol buffer once. The bead samples were dissolved in Laemmli sample buffer supplemented with 10 mM DTT and boiled for 5 min at 95° C. before loading on a 4-12% Bis-Tris NuPAGE gels. Proteins were transferred onto polyvinylidene fluoride (PVDF) membranes (Millipore #05317) by using the iBlot2 Dry Blot system (Thermo Fisher Scientific) following to instruction manual, and detected with specific antibodies (anti-HDAC6 (D2E5, Cell Signaling Technology), 1:1,000, GFP (JL-8, Takara), 1:1,000, and a-Tubulin (Abcam ab4074) 1:1,000).

IAV Infection Assay (in Nanobody Expressing Cells)

A549 cells stable expressing the HDAC6 ZnF nanobodies were infected with IAV X-31 (H3N2) strain. The cells were trypsinised and fixed in 4% FA at 5.5 hours post infection. The cells were stained for FACS analysis in FACS buffer (PBS, 0.1% BSA) containing 0.1% Saponin. The primary antibody used was mAb HB-65 (anti-nucleoprotein, ATCC), 1:200, and secondary was goat anti-mouse IgG Alexa Fluor 647 (Invitrogen), 1:2500. Antibodies were incubated for 30 min at room temperature. The cells were washed by centrifugation at 2,500 rpm for 5 min, resuspended in 100 μL of FACS buffer and analysed using Novocyte Flow Cytometer (Aceabio). The fcs files were analysed using Flow Jo version 10.3.0.

Selection and Screening of DARPins

To generate DARPin binders against HDAC6-ZnF, the biotinylated target protein (see below) was immobilized on MyOne T1 streptavidin-coated beads (Pierce). Ribosome display selections were performed essentially as described (Dreier and Plückthun, 2012). Selections were performed over four rounds with decreasing target concentration and increasing washing steps to enrich for binders with high affinities. After four rounds of selection, the enriched pool was cloned into a bacterial pQIq-based expression vector as fusion with an N-terminal MRGSH₈- and C-terminal FLAG tag. After transformation into E. coli XL1-blue 380 single DARPin clones were expressed in 96 well format and lysed by addition of Cell lytic B reagent (Sigma), Lysozyme and Pierce nuclease. These bacterial crude extracts of single DARPin clones were subsequently used in a Homogeneous Time Resolved Fluorescence (HTRF)-based screen to identify potential binders. Binding of the FLAG-tagged DARPins to streptavidin-immobilized biotinylated HDAC6-ZnF was measured using FRET (donor: streptavidin-Tb cryptate, 610SATLB; acceptor: anti-FLAG-d2, 61 FG2DLB; both Cisbio). Experiments were performed at room temperature in white 384-well Optiplate plates (PerkinElmer) using the Taglite assay buffer (Cisbio) at a final volume of 20 μL per well. FRET signals were recorded after an incubation time of 30 minutes using a Varioskan LUX Multimode Microplate (Thermo Scientific) with the following settings: Delay time: 60 μs, integration time: 200 μs, measurement time: 1,000 ms, dynamic range: automatic. HTRF ratios were obtained by dividing the acceptor signal (665 nm) by the donor signal (620 nm) and multiplying this value by 10,000 to derive the 665/620 ratio. The background signal was determined by using reagents in the absence of DARPins. From this result, potential binders were identified (Table S1)

Expression and Purification of Biotinylated his-Avi-HDAC6 ZnF (1108-1215), HDAC6 ZnF (1108-1215) and DARPin F10

E. coli BL21 (DE3), transfected by pOPINF-His-Avi-HDAC6 ZnF (in this case, bacteria was co-transfected with pet21a-BirA expressing plasmid, pOPINF-His-HDAC6 ZNF or DARPin F10 plasmid (pQiq_K_MRGS_His10-HA-3C-1766_F10), was cultured first in 50 ml LB medium overnight at 37° C., then 10 ml medium was transferred to 1 L 2xYT medium for continuous culturing in 2.5 L flask. When OD=0.6 was reached, IPTG was added into the 1 L medium (final concentration 1 mM) and the temperature was reduced to 17° C. (to induce ZnF protein biotinylation, D-biotin was added to 2xYT medium to a final concentration of 20 μM). Cultures were grown further for 18 hrs and bacteria were collected by centrifugation (4000 rpm, 15 mins, 4° C.). The pellet was frozen at −80° C. All constructs were purified as follows.

The bacterial pellet was lysed with an ultrasonic sonicator (15 cycles, one cycle=20 secs ON, 40 secs OFF) to completely break the bacteria. The cell lysate was dissolved in Ni column loading buffer (buffer 1, containing 20 mM Tris pH=7.5, 500 mM NaCl, 10 mM imidazole, supplied with protease inhibitor 1 mM PMSF). The cell debris were separated from the protein by High-speed-centrifugation (17000 rpm, 1 hr, 4° C.). After filtering (0.45 μM filter, Merck #SLHVM33RS) the supernatant, the protein solution was loaded onto a HisTrap column (#GE17-5248-01) using a peristaltic pump at a flow rate of 5 ml/min. The column was washed with 4 to 5 column volume (CV) of buffer 1. To elute the target protein, we used a gradient elution by the AKTA system. The elution buffer (buffer 2, containing 20 mM Tris pH=7.5, 500 mM NaCl, 250 mN imidazole) was used together with the buffer 1 to generate the gradient.

Eluted protein was digested by 3C protease (obtained from FMI protein facility) in dialysis buffer (20 mM Tris pH=7.5, 500 mM NaCl) at 4° C. Digested protein was re-loaded onto a HisTrap column to remove the His tag. His-Avi-ZnF protein was not treated with 3C protease, but loaded directly to the gel filtration step. The flow-through was collected and protein purity was determined by SDS-PAGE. Different fractions (with a purity >50%) from gradient elution were combined and applied onto an ion exchange column. Here, the protein solution was diluted 5 times with 20 mM Tris pH=7.5 buffer and loaded onto a 5 ml pre-pack HiTrap Q HP(#GE29-0513-25) column. Protein was eluted over a 25 min period with a gradient generated by the AKTA system, by mixing buffer A (20 mM Tris pH=7.5, 100 mM NaCl, 2 mM Tcep) and buffer B (20 mM Tris pH=7.5, 1 M NacL, 2 mM Tcep). Eluted target protein was concentrated and loaded onto Gel filtration system, using either a Superdex® 200 Increase 10/300 GL (#GE28-9909-44) or HiLoad 16/600 Superdex 200 pg (#GE28-9893-35) column. After Gel filtration, the separated target protein was flash-frozen with liquid nitrogen in Gel filtration buffer (20 mM Tris pH=7.5, 100 mM NaCl, 1 mM TCEP). Protein concentration was determined by its absorbance at 280 nm.

Pull-Down Assay to Identify Inhibitory DARPin

10 μg Purified His-HDAC6 ZnF (1108-1215) was immobilized with 20 μL Ni-NTA agarose (Promega #30210) slurry at 4° C. for 30 mins. Subsequently, 20 μg DARPin protein and 10 μg Ub were added to the Ni-NTA-ZnF solution at same time (in FIG. S1b, they were mixed as described in the figure legend). All proteins were diluted in Ni-NTA loading buffer (20 mM Tris pH=7.5, 100 mM NaCl, 10 mM imidazole). The reaction volume was 500 μL, incubation was at 4° C. on rotator for 30 mins. After incubation, the beads were washed 3 times with washing buffer (20 mM Tris pH=7.5, 150 mM NaCl). The supernatant was removed after spinning down the beads (500 g, 2 mins), and 20 μL 1×LDS sample buffer (Invitrogen #NP0007) was added to each reaction. Following heating at 80° C. for 10 mins, all the samples were loaded onto NuPAGE 4-12% gradient gels. The proteins were visualized with Instant Blue reagent (expedeon #ISB1L).

Isothermal Titration Calorimetry (ITC) for Determining Affinity

The experiment was performed on a MicroCal VP-ITC machine. Protein HDAC6 ZnF (1108-1215) and DARPin F10 were purified as described, and Ub was purchased from BostonBiochem(Cat #U-100H). All the proteins were dialyzed in ITC buffer (10 mM Tris pH=7.5, 100 mM NaCl) for 3 hrs at 4° C. before the experiment. We titrated 0.2 mM DARPin F10 protein (in the syringe) to 0.02 mM HDAC6 ZnF (1108-1215) protein in the cell (for determination of Ub and ZnF, 0.25 mM Ub was titrated to the 0.01 mM ZnF). The curve and statistics were done with the MicroCal ITC Origin Analysis software.

LC-MS Analysis for DARPin F10 Interacting Protein

5 μg pcDNA3.1-GFP-DARPin F10 and pcDNA3.1-GFP-DARPin E3_5 were transfected into A549 cells; for each construct 3×10 cm dishes were used, each containing 2×10\hat{ }6 cells in 10 cm dishes. For each sample 15 μL Lipofectamine 3000 were used and the cells were cultured for 2 days to allow for sufficient expression. After collecting the cells, each dish was lysed with 500 μL CoIP buffer (10 mM Tris pH=7.5, 150 mM NaCl, 0.5 mM EDTA, 0.5% NP-40). The lysate was centrifuged at 14,000 rpm and 10 mins, 4° C. Supernatant was transferred to a new tube, and the protein concentration was determined by Bradford Assay (Biorad #5000006). Same amount of protein lysate (1.0 to 1.5 μg) was incubated with 10 μL GFP_Trap M beads (Chromotek #gtm-20) on rotator at 4° C., overnight. In the following morning, the beads were washed with washing buffer (20 mM Tris pH=7.5, 150 mM NaCl) 3 times.

To digest the protein on the beads, we used 10 μL Lys-C (0.2 μg/μL in 50 mM Hepes pH 8.5) and 50 μL digestion buffer (3 M GuaHCl, 20 mM EPPS pH 8.5, 10 mM CAA, 5 mM TCEP) to make master mix, and added 6 μL of this mixture to the beads from each 10 cm dish. After short spin down (<1000 g) and 4 hours incubation at 37° C., we added 17 μL 50 mM HEPES pH 8.5 as well as 1 μL 0.2 μg/μL trypsin to further digest the protein at 37° C. overnight. Next morning, 1 more μL trypsin was added to the solution for 6 additional hours digestion. Then the sample was processed at the FMI Protein Analysis facility for mass spectrometry. The data was analyzed by software Perseus (version 1.5.2.6). And protein was annotated with human (v2017-04) database.

RNA Seq Analysis for DARPin F10 Impact on Cellular Gene Expression

The A549 cells were transfected as described in above (same as in section “LC-MS analysis”). Each plasmid was transfected into 3×10 cm dish cells. After 2 days in culture, 1.5 million GFP positive cells were isolated from each dish by FACS (that is, 3×1.5 million cells for one transfected DARPin), and total RNA was extracted with the RNA extracting Kit (QIAGEN #74004). The samples were further processed at the FMI genomics Facility and sequenced on a Hi-Seq instrument; the results were analysed with R-Studio.

Generation of A549 Cells Expressing a Conditionally Degradable DARPin F10

Plasmid Plenti-Puro-Flag-HA-DARPin F10-FKBP^{F36V} and Plenti-Puro-Flag-HA-FKBP^{K36V} (each 20 μg) were co-transfected with packaging plasmids (expressing tat, rev, gag, vsv-g, each at 1 μg) together with 75 μL FuGENE HD reagent (Promega #E2311) in 0.5 ml Opti-MEM medium (Sigma #31985062). After 25 mins incubation at room temperature, the mix was added to 293T cells in 10 cm dish, seeded one day before with 1.6 million cells per dish. Cells were cultured at 37° C. for 3 days; the medium was collected and filtered with 0.45 μm filter (Merck #SEIM003 M00), and 1× LentiX concentrator (Takara #631231) was added (⅓ of the supernatant volume). The mixture was incubated at 4° C. for 30 mins and the lentivirus was precipitated by centrifugation at 1500 g, 45 mins, 4° C. The pellet was re-suspended in 500 μL Opti-MEM (Gibco #31985062).

The re-suspended lentivirus pellet was added to the culture medium of A549 cells (10 cm dish, 0.6 million WT A549 cells seeded per dish one day before). Two days later the medium was changed to DMEM supplied with puromycin (final concentration 2 μg/ml). Puro-resistant cells were selected for 2 days and then single-cell sorted into 96 wells plates. After 1 month culturing, clones were expanded and analyzed by western blot with HA antibody (Abcam #18181) to identify the cell lines expressing HA-DARPin F10-FKBP^{F36V} or HA-FKBP^{F36V}.

Split-GFP Assay

Plasmid pcDNA3.1-GFP(1-9), pcDNA3.1-GFP(10)-ubiquitin, pcDNA3.1-GFP(11)-HDAC6 ZnF (1108-1215)/1182 mutant and pcDNA3.1-mRuby were co-transfected with FuGENE reagent (using the manufacturer's protocol) (Promega #E2311) together to 0.5 million 293T cells in 6 well plate, with a molarity ratio of 1:1:1:1 (1 μg for pcDNA3.1-GFP(1-9), other plasmids were adjusted accordingly). After 2 days culturing at 37° C., the GFP signal was visualized under wide field microscopy (Zeiss Z1). mRuby expression served as a transfection control.

To investigate DARPin interference, non-fluorescent tagged DARPin F10 and DARPin A10 plasmid (1 μg for both) were transfected together with the plasmids mentioned above. Visualization procedures were the same.

IAV Uncoating Assay

A549 cells were seeded on glass slides and, after reaching ca. 70% confluence, incubated at 4° C. with IAV X31 (H3N2, MOI=30 PFU/cell) for 1 h to synchronize infection. After that, the cells were incubated at 37° C. for 3 h and fixed with paraformaldehyde 4% for 15 min. For immunofluorescence, cells were permeabilized with 0.5% Triton X-100 in PBS for 5 min and incubated overnight in 1% BSA with M1-specific murine monoclonal antibody (ATCC #HB64, 1:4000). Cells were washed with PBS and incubated for 1 h with Alexa Fluor 488 goat anti-mouse (IgG) (H+L) (Thermo Fischer; 1:2000, 1% BSA) for 1 h at room temperature. Nuclei were stained for 5 min with DAPI (1:1000 in PBS). Glass slides were examined using spinning disk confocal scanning unit. Alex Fluor 647 NHS ester Tris was used to stain the total protein to visualize the cell body.

The mean fluorescence green intensity (MFI) was quantified using ImageJ. One representative out of three independent experiments is shown. For all panels, error bars represent standard deviation of the pictures analyzed (approximately 40 cells per picture and more than 200 cells in total) and statistical significance was determined by one-way ANOVA (ns (non-significant)=P>0.05; *P≤0.05; **P≤0.01; ***P≤0.001).

Virus Growth Curve

A549 cells were infected in 6 well/plates with Influenza virus (Virapur, H3N2, purified Influenza A/X31, #B1707C) at 37° C. in infection medium (DMEM 0.2% BSA, 2 mM L-glutamine and 1 μg/ml of TPCK-treated trypsin). For single cycle infection assays, cells and viruses were pre-incubated at 4° C. for 1 h and the cells were washed with PBS before incubation at 37° C. for the indicated time points. Viruses from the harvested supernatants were quantified using plaque assay in MDCKII cells with 2% agar overlay.

A Zika virus stock from a low passage clinical isolate of Asian lineage (ZIKV PRVABC59) passaged on Vero cells and purified by ultracentrifugation was used to infect A549 cells in 24 well/plates at 37° C. with in DMEM 10% FCS. Virus titers collected from the culture supernatant were determined on Vero cells (ATCC #CCL-81) and expressed as 50% tissue culture infective dose per ml (TCID50/ml) using the Reed and Muench method.

Immunofluorescence for Zika Virus

For detection of Zika virus protein on infected cells 72 hpi, the A549 cells were washed with PBS, fixed with 4% PFA and incubated with anti-E protein Flavivirus group antibody 4G2 in PBS supplemented with 1% BSA for 1 h at 37° C. Cells were washed with PBS and incubated for 1 h with Alexa Fluor 488 goat anti-mouse (IgG) (H+L) (Thermo Fischer; 1:2000, 1% BSA) for 1 h at room temperature. Nuclei were stained for 5 min with DAPI (Thermo Fischer, 1:1000 in PBS).

Quantification and Statistical Analysis

Peptides of raw DARPin F10 data from the mass spectrometers were identified and quantified by MQ version 1.5.3.8. For data searches the search engine Andromeda was used. A costume DB with the DARPin_F10 and E3_5 sequence combined with the human sub-set of the UNIPROT DB and the contaminant library from MaxQuant was searched. Results were filtered with a FDR of 1%. Details of specific statistical tests and experimental design for immunofluorescent experiments are given in the relevant figure legends. All Virus growth curve experiments were performed in triplicate (n=3) for technical relocation. Each experiment was independently repeated 2 to 3 times to consolidate the conclusion. To analyze the data, two-way ANOVA test (using the Geisser-Greenhouse Correction) was performed with GraphPad Prism 8; Abbreviations for p values are as follows: p<0.05=*, p<0.01=**, p<0.001=***, p<0.0001=****; significant P values were shown.

1. A recombinant binding protein comprising at least 90 consecutive amino acids of SEQ ID NO:1, wherein said recombinant binding protein specifically binds to HDAC6 and blocks the ubiquitin-engaging zinc finger domain of HDAC6.

2. The recombinant binding protein of claim 1, comprising at least 120 consecutive amino acids of SEQ ID NO:1,

3. A recombinant binding protein that competes for binding to HDAC6 with the recombinant binding protein of claim 1 and blocks the ubiquitin-engaging zinc finger domain of HDAC6.

4. An isolated nucleic acid encoding a recombinant binding protein according to claim 1.

5. The isolated nucleic acid of claim 4, wherein said isolated nucleic acid comprises the nucleic acid sequence of SEQ ID NO:2

6. An expression cassette comprising the isolated nucleic acid of claim 4, wherein said isolated nucleic acid is operatively linked to genetic elements allowing and/or controlling its expression in target cells.

7. The expression cassette of claim 6 wherein one of the elements controlling the expression of the isolated nucleic acid allows for conditional expression of said isolated nucleic acid.

8. A vector comprising the expression cassette of claim 6.

9. (canceled)

10. (canceled)

11. A pharmaceutical composition comprising a recombinant binding protein according to claim 1 and optionally a pharmaceutically acceptable carrier and/or diluent.

12. A method for treating a viral infection in a subject characterised in that a therapeutically effective amount of a recombinant binding protein according to claim 1 is administered to said subject, optionally in a pharmaceutical composition comprising said recombinant binding protein.

13. The method of treatment of claim 10, wherein the virus is an enveloped virus optionally selected from the group consisting of an influenza virus, a zika virus or an ebola virus.

14. (canceled)

15. The recombinant binding protein of claim 1, wherein said recombinant binding peptide binds to HDAC6 with a K_{D} below 4 μM.

16. A pharmaceutical composition comprising an isolated nucleic acid according to claim 4 and optionally a pharmaceutically acceptable carrier and/or diluent.

17. A pharmaceutical composition comprising an expression cassette according to claim 6 and optionally a pharmaceutically acceptable carrier and/or diluent.

18. A pharmaceutical composition comprising a vector according to claim 8 and optionally a pharmaceutically acceptable carrier and/or diluent.

19. A method for treating a viral infection in a subject characterised in that a therapeutically effective amount of an isolated nucleic acid according to claim 4 is administered to said subject, optionally in a pharmaceutical composition comprising said isolated nucleic acid.

20. The method of treatment of claim 19, wherein the virus is an enveloped virus optionally selected from the group consisting of an influenza virus, a zika virus or an ebola virus.

21. A method for treating a viral infection in a subject characterised in that a therapeutically effective amount of an expression cassette according to claim 6 is administered to said subject, optionally in a pharmaceutical composition comprising said isolated nucleic acid.

22. The method of treatment of claim 21, wherein the virus is an enveloped virus optionally selected from the group consisting of an influenza virus, a zika virus or an ebola virus.

23. A method for treating a viral infection in a subject characterised in that a therapeutically effective amount of a vector according to claim 8 is administered to said subject, optionally in a pharmaceutical composition comprising said isolated nucleic acid.

24. The method of treatment of claim 23, wherein the virus is an enveloped virus optionally selected from the group consisting of an influenza virus, a zika virus or an ebola virus.
